(19) United States
(12) Patent Application Publication
(10) Pub. No.: US 2009/0004155 A1
(43) Pub. Date: Jan. 1, 2009

(54) Method for treating a mammal having damaged myocardium tissue

(76) Inventors: Patrick J. Casey, Kumeu (NZ); Kerri Fry, Kumeu (NZ); Richard Fry, Kumeu (NZ)

Correspondence Address: PATRICK R. SCANLON;PRETI FLAHERTY BELIVEAU & PACHIOS LLP, ONE CITY CENTER, PORTLAND, ME, 04112-9546, US

(21) Appl. No.: 12/072,509

(22) Filed: Feb. 26, 2008

Related U.S. Application Data
(60) Provisional application No. 60/903,425, filed on Feb. 26, 2007.

(51) Int. Cl. A61K 35/34; A61P 9/00

(52) U.S. Cl. 424/93.7

(57) ABSTRACT

A method for treating a mammal having damaged myocardium tissue includes harvesting a tissue sample from the mammal, growing myocardium cells from the tissue sample, and transplanting the myocardium cells into the damaged tissue. Growing the myocardium cells from the tissue sample can be accomplished by breaking the tissue sample into fragments, placing the fragments into a culture vessel, inducing at least some of the fragments to adhere to the culture vessel, and supplying the fragments with nutrients so that myocardium cells contained therein divide and grow.

### CROSS REFERENCES TO RELATED APPLICATIONS

This application claims the benefit of U.S. Provisional Application No. 60/903,425, filed Feb. 26, 2007.

### BACKGROUND OF THE INVENTION

Heart disease resulting from damaged myocardium tissue is a common ailment in many mammals, including humans. Treatment options for damaged myocardium tissue are limited and often ineffective.

Accordingly, it would be desirable to develop improved methodologies for treating mammals having damaged myocardium tissue.

### SUMMARY OF THE INVENTION

The above-mentioned need is met by the present invention, one embodiment of which includes a method for treating a mammal having damaged myocardium tissue that includes harvesting a tissue sample from the mammal, growing myocardium cells from the tissue sample, and transplanting the myocardium cells into the damaged tissue. Growing the myocardium cells from the tissue sample can comprise breaking the tissue sample into fragments, placing the fragments into a culture vessel, inducing at least some of the fragments to adhere to the culture vessel, and supplying the fragments with nutrients so that myocardium cells contained therein divide and grow.

The present invention and its advantages over the prior art will be more readily understood upon reading the following detailed description.

### DETAILED DESCRIPTION OF THE INVENTION

The present invention generally relates to an in vitro method for growing myocardium cells from a tissue sample taken from a mammal or other organism having damaged tissue structure, and transplanting the grown cells back into the same mammal or organism to assist in repairing the damaged tissue structure. The method applies in particular to repairing damaged myocardium tissue. In general, the method comprises harvesting a tissue sample from a patient and using the tissue sample to grow adult myocardium cells under specific culture conditions. For example, cells originating from a patient's heart tissue can form myocardium cells in vitro. While the present invention is applicable to mammals, and particularly eutherian mammals, it should be noted that the present invention is not limited to mammalian cells.

In one embodiment, the method is performed on a mammal having damaged heart tissue (i.e., a discrete core lesion). The first step is to harvest one or more tissue samples from the injured mammal using a minimally invasive biopsy technique. The samples can be taken from the damaged heart tissue itself, but to ensure there is no further damage caused to the injured tissue, the samples can be taken from a surrogate heart tissue source. The surrogate tissue source should be a tissue that is similar to the damaged tissue and will form similar cell structures in vitro. Samples can be taken from the mammal's heart tissue using a spring-loaded biopsy instrument under local anesthesia.

The harvested samples are transported to the laboratory at 4 degrees Celsius for further processing. Cellular growth is stimulated by breaking the tissue samples down into fragments. Fragmentation may be accomplished with any suitable technique, including but not limited to dissection, chemical digestion, and physical digestion. The fragments are placed in a culture vessel and induced to attach or adhere to the vessel, which will allow cells to grow and divide. The attached tissue fragments are then supplied with nutrients (for example, but not limited to, immersion in specific cell culture media under predetermined conditions) to induce the division/multiplication of adult myocardium cells. In one embodiment, the cells are placed in a modified “hanging drop” culture system wherein the fragments are placed into a culture flask with only enough media to keep tissue moist, not suspended. This flask is then flipped over (or partially tipped) and a small amount of media is added to the bottom to keep the tissue fragments humidified. With the flask inverted, the fragments are stressed by gravity but tend to remain in contact with the flask surface due to the moisture from the media.

Cells are grown in the laboratory until there are enough cells to transplant back into the core lesion, at which point cells can be harvested. For example, myocardium cells are harvested using a Trypsin/EDTA incubation technique after a sufficient passage of time, such as 14 days. Between 10-20 million cells can be harvested.

The harvested myocardium cells are injected back into the core lesion of the same injured mammal, thus providing cells of the correct type that avoid rejection. The injected cells will aid in the repair of the damaged tissue. Typically, the cells are trypsonised prior to implantation, loaded into a syringe and injected back into the same mammal that the samples came from (autotransplantation).

While specific embodiments of the present invention have been described, it should be noted that various modifications thereto can be made without departing from the spirit and scope of the invention as defined in the appended claims.

What is claimed is:

1. A method for treating a mammal having damaged myocardium tissue, said method comprising:
harvesting a tissue sample from said mammal;
growing myocardium cells from said tissue sample; and
transplanting said myocardium cells into said damaged tissue.

2. The method of claim 1 wherein said tissue sample is harvested from said mammal's heart.

3. The method of claim 1 wherein growing myocardium cells from said tissue sample comprises:
breaking said tissue sample into fragments;
placing said fragments into a culture vessel;
inducing at least some of said fragments to adhere to said culture vessel; and
supplying said fragments with nutrients so that myocardium cells contained therein divide and grow.

4. The method of claim 3 wherein inducing at least some of said fragments to adhere to said culture vessel includes:
placing said fragments into said culture vessel;
adding enough media to keep said fragments moist but not suspended;
flipping said culture vessel over; and
incubating said fragments.

5. The method of claim 3 wherein breaking the tissue sample into fragments includes dissection.

6. The method of claim 3 wherein breaking the tissue sample into fragments includes chemical digestion.

7. The method of claim 3 wherein breaking the tissue sample into fragments includes physical digestion.

8. The method of claim 3 further comprising removing myocardium cells from said culture vessel after a sufficient number of cells have grown.

9. The method of claim 8 wherein cells are removed via Trypsin/EDTA incubation.
